(19) United States
(12) Patent Application Publication
(10) Pub. No.: US 2012/0130174 A1
(43) Pub. Date: May 24, 2012

(54) Rapid Endoscopic Gastrointestinal Irrigation System for Gastrointestinal Bleeding

(76) Inventor: Harry W.R. Simmons, Riner, VA (US)

(21) Appl. No.: 12/675,497

(22) PCT Filed: Aug. 31, 2007
(86) PCT No.: PCT/US07/77393

§ 371 Date: Sep. 22, 2010

(51) Int. Cl. A61B 1/015

(52) U.S. Cl. 600/159

(57) ABSTRACT

The present invention relates to surgical instruments used in combination with endoscopes during gastrointestinal procedures for clearing a surgical site of unwanted obstructions, such as blood and/or clots. More particularly, the irrigation devices according to the invention comprise a handle comprising valves for controlling fluid flow, at least one channel for transporting fluid, and a connection port adapted to communicate directly or indirectly with an endoscope for delivering fluid to and receiving fluid from and through the endoscope. The irrigation devices and systems incorporating them can be used in any endoscopic gastrointestinal surgical procedure to clean, clear, or evacuate an area of the gastrointestinal tract, such as the stomach, in cases of accidental poisoning, drug overdose, or gastrointestinal bleeding. The irrigation device according to the present invention is capable of clearing a stomach within seconds or up to 1-2 minutes.

### CROSS-REFERENCE TO RELATED APPLICATIONS

This application is a U.S. National Stage application of International Patent Application No. PCT/US07/77393, filed on Aug. 31, 2007, which has been published as International Publication No. WO2009/029106 on Mar. 5, 2009, the disclosure of which is incorporated by reference herein in its entirety.

### BACKGROUND OF THE INVENTION

1. Field of the Invention

The present invention relates generally to the field of medical devices. More particularly, the present invention relates to surgical instruments that can be used in endoscopic procedures, such as endoscopic gastrointestinal surgery, for clearing a surgical site of unwanted obstructions, such as blood and/or clots.

2. Description of Related Art

Current methods for cleaning, clearing, or stopping bleeding during endoscopic gastrointestinal procedures typically involve manually irrigating the surgical site with saline, then aspirating the saline and other debris manually away from the site. Current manual evacuation methods include injecting a saline solution into the patient at the surgical site using a syringe in combination with tubing, then evacuating the fluid by reversing the movement of the plunger of the syringe. One such system is the Kimberly-Clark Easi-Lav gastric lavage system. The disadvantages to this system, however, include only being able to deliver then aspirate small amounts of saline and debris. Using such manual evacuation techniques, the irrigation/evacuation steps must be performed numerous times successively to clear the site adequately, which can take on the order of up to a half hour or more to evacuate a large area, for example, the stomach.

Complicating matters, such evacuation systems are typically used with a separate scope system to determine whether the surgical site has been adequately cleared. For upper gastrointestinal procedures, the separate scope, which is typically housed within a thin flexible tube, is usually inserted, removed, and reinserted through the patient's throat after each cleaning or evacuation step. This process of irrigating/evacuating then checking with a scope must be repeated until the surgeon is satisfied with the clearing overall. Numerous disadvantages exist with such techniques, including to name a few: 1) an increased risk of patient infection and/or discomfort by the constant insertion into and removal from the patient of various instruments during surgery; (2) increased surgical time; (3) the patient is under the influence of anesthesia for prolonged periods; and (4) an increase in costs for longer, complicated, more involved surgery.

Because of the lack of adequate surgical instruments for endoscopic cleaning, clearing, or stopping an active bleed at a surgical site, surgeons have been known to improvise by modifying instruments typically used for other purposes. Improvising during surgery, however, is often time consuming for the surgeon, which is not desirable especially in an emergency situation. Tools and instruments at the surgeon's disposal also are typically not adaptable to fit the surgical situation precisely. In addition, because the instruments available to the surgeon are made for different purposes and are not configured to cooperate with each other, the surgeon is still faced with the predicament of introducing, removing, and reintroducing different instruments into and out of the patient during surgery.

Thus, there exists a need for a streamlined approach for performing efficient endoscopic clearing of a gastrointestinal surgical site of unwanted matter and/or stopping or controlling an active bleed. In particular, there exists a need for an automated endoscopic surgical instrument capable of quickly irrigating and evacuating a gastrointestinal cavity of a patient, for example, to control or stop gastrointestinal bleeding.

### SUMMARY OF THE INVENTION

The devices and systems of the present invention are suitable for use during any endoscopic procedure, such as endoscopic gastrointestinal procedures. For example, during an upper gastrointestinal procedure the irrigation system according to the invention can be used to clear a surgical site of unwanted matter, such as blood or clots, which may be interfering with performance of the surgery. The system according to the invention can also be used to identify a source of bleeding and to control the bleeding so that a surgical procedure can be performed and/or the cause of the bleed can be repaired.

The present invention includes devices, systems, and methods for endoscopic clearing of a surgical site of unwanted matter. The present invention can be used in any surgical procedure, for example, gastrointestinal irrigation, to clean, clear, or evacuate an area, such as the stomach, in cases of accidental poisoning, drug overdose, or gastrointestinal bleeding. Depending on the severity of the evacuation needed, the devices according to the invention are capable of clearing a stomach sufficiently within seconds or up to 1-2 minutes. This is substantially faster than existing techniques, which can take up to a half hour or longer. For example, the device according to the invention can clear one unit of blood in about 5 min. Such efficiencies lead to better results for the patient, which typically equates to less time in surgery, faster injury repair, and/or less blood loss by the patient. In cases of gastrointestinal tract bleeding, for example, the present invention can be used to identify and control the bleeding quickly to enable the surgeon to repair the injury faster, which in some cases may translate to a total blood loss difference in the patient on the order of 1-4 units of blood. The present invention, thus, provides devices, systems, and methods for endoscopic visualization, control, or repair of an active bleeding site within the body, such as the gastrointestinal tract, in a fraction of the time that current methods provide.

Included within the scope of the present invention is an irrigation device for endoscopic surgery comprising a handle having valves for controlling fluid flow; at least one channel for transporting fluid; and a connection port adapted to communicate with an endoscope for delivering fluid to and through and receiving fluid from the endoscope. The irrigation devices can be used for gastrointestinal procedures. The irrigation device can be configured to cooperate with any endoscope. Examples of endoscopes include gastroscopes, colonoscopes, sigmoidoscopes, and enteroscopes to name a few. The irrigation device is typically compatible with endoscopes that are intended for use through a natural or existing orifice of a patient's body, however, the irrigation device can also be configured to be compatible with a laparoscope, which is intended to be used through a surgical incision introduced to a patient's body. Further, the endoscopes can be diagnostic or therapeutic in nature, meaning the endoscopes can be used to examine and diagnose a patient and/or to treat a patient, such as by performing a biopsy or cauterization.

In embodiments, the irrigation devices according to the invention can comprise at least three channels, each for transporting fluid from a fluid source to the irrigation device or for transporting fluid to a waste container from the irrigation device. For example, the irrigation devices can comprise a first channel capable of transporting irrigation fluid to the irrigation device, a second channel capable of transporting pressurized gas to the irrigation device, and a third channel capable of transporting aspirated fluid from the irrigation device.

The irrigation devices are capable of transporting any irrigation fluid or wash fluid, including saline and water, and any pressurized gas, including air and carbon dioxide or combinations thereof.

The irrigation devices according to the invention can be incorporated into systems for endoscopic gastrointestinal surgery and/or gastrointestinal irrigation systems, as well as included in methods of gastrointestinal surgery, methods of gastrointestinal irrigation, and methods of providing instruments for endoscopic procedures.

Systems for endoscopic surgery are also included in the invention. Such systems comprise: 1) an irrigation device comprising a handle comprising valves for controlling fluid flow, at least one channel for transporting fluid, and a connection port adapted to communicate with an endoscope for delivering fluid to and through and receiving fluid from the endoscope; 2) an endoscope; and 3) a supply of fluid and suction. Such systems can be used for endoscopic gastrointestinal procedures. In embodiments, the connection port of the irrigation device may be adapted to communicate either directly or indirectly with the endoscope. For example, a connector, fitting, or other adapter (including any combination of tubing, connectors, fittings, and adapters) could be used to join the irrigation device to the endoscope. The structure for connecting the irrigation device to the endoscope need not conform to any particular configuration. For example, the intermediate structure may comprise a Y-shaped adapter, wherein one channel of the Y-shaped structure is used for the passage of fluids to and from the irrigation device and the other channel is used to introduce auxiliary therapeutic instruments into the patient through the endoscope, such as biopsy forceps or an instrument for cauterization.

Surgical systems in accordance with the invention preferably comprise a portable source of fluid and a portable storage for waste disposal. For example, in embodiments, the systems according to the invention can comprise a portable base unit which houses the fluid source(s) and waste receptacles and which can comprise power source(s).

Included within the invention are systems for endoscopic irrigation, such as gastrointestinal irrigation, comprising: 1) an irrigation device comprising a handle comprising valves for controlling fluid flow, at least one channel for transporting fluid, and a connection port adapted to communicate directly or indirectly with an endoscope for delivering fluid to and through and receiving fluid from the endoscope, and 2) a supply of fluid and suction. Such irrigation systems are capable of transporting irrigation fluid (e.g., saline or water), pressurized gas (e.g., carbon dioxide or air), and suction (e.g., vacuum). The irrigation systems can further comprise a Y-shaped adapter for connecting the irrigation device to the endoscope for therapeutic purposes. The term “adapter” as used in the context of this application is meant to encompass any intermediate structure, or combinations of structures, that is used between the irrigation device and the cooperating endoscope. Means for connecting the intermediate structure to either or both of the irrigation device and the endoscope may be incorporated into any or all of the intermediate structure, the irrigation device, and/or the endoscope.

The irrigation devices, surgical systems, and methods comprising them can further comprise a pressure sensor for monitoring fluid pressure.

Portable irrigation systems, including portable fluid supplies, waste receptacles, and power source(s), such as a battery, are also included within the invention.

Methods of providing instruments for endoscopic irrigation or endoscopic surgery, including gastrointestinal irrigation and/or endoscopic gastrointestinal surgery, are also encompassed by the invention. For example, methods of providing instruments, such as an irrigation device and/or irrigation system according to the invention, to surgeons, other medical practitioners, or institutions for performing endoscopic patient procedures form a part of this invention. More particularly, the invention includes performing surgery with, supplying, or providing irrigation devices and systems of the invention for medical procedures. Supplying or providing the devices and systems encompasses making the devices and systems available for use in endoscopic procedures.

The rapid endoscopic irrigation devices and systems according to the invention are designed to be compatible with and can comprise conventional endoscopes, for example, endoscopes used in gastrointestinal surgery. In one embodiment, the irrigation device is configured to be a component of an endoscope and is designed to be held and operated by the surgeon in one hand, while the cooperating endoscope is concurrently held and operated by the surgeon in the other hand. The irrigation device can be connected directly to the endoscope or can be indirectly connected by way of a tubing adapter having one or more channels, such as a Y-shaped adapter. In embodiments, multiple adapters can be provided in a kit, so as to provide multiple options for the surgeon, such as to provide the option of introducing auxiliary instruments into the patient through the endoscope. In embodiments, kits and adapters preferably comprise, or are configured to cooperate with, quick-connect/disconnect fittings for communication with the port of the irrigation device as well as with the ports of various endoscopes. Kits and adapters can comprise, for example, multiple fittings to present the surgeon with various options for using multiple types of endoscopes. In such kits, the fittings can be incorporated into the intermediate structure (adapter), can be incorporated into the irrigation devices and/or endoscopes, or can be separate structure.

Several systems are provided by the present invention. In one system, there is an endoscope, an irrigation system, and a base supply unit. The supply unit, which can be portable, provides for electricity for powering the complete system as well as reservoirs for the fluid supplies, such as saline, water, carbon dioxide, and air, and means for suction.

The irrigation devices according to the present invention generally comprise a means for holding the irrigation device (such as a handle), means for controlling fluid flow (such as valves, e.g., push-button valves), and one or more means for transporting fluids (such as fluid channels). The fluids are transported through the irrigation device under pressure and means for controlling fluid flow, such as valves or a valve system, is incorporated into the device.

In the context of this invention, fluid is any matter that has a tendency to flow, e.g., matter comprising molecules that move freely past one another. Fluid includes any gas or liquid, including water, saline, air, and carbon dioxide to name a few.

The fluid channels of the irrigation devices are comprised of tubing, which lead from a fluid source or a fluid reservoir to the irrigation device. In one embodiment, three fluid channels or tubes lead from each of an irrigation fluid source, a pressurized gas source, and a waste reservoir.

The tubing of the irrigation devices is connected to the fluid sources and the waste receptacle (preferably housed on or in a base supply unit) by any means, such as a quick-connect/disconnect means, which allows for easy and efficient set up of the irrigation device with the fluid supply and waste containers. More particularly, one tube is connected to a pressurized gas source (such as carbon dioxide or air), a second tube is connected to a saline or water source, and a third tube is connected to a waste receptacle. In embodiments, the three separate tubing channels can be incorporated into a multi-tube connector, which can cooperate with a complimentary multi-tube connector at the connection of the irrigation device with the base supply unit. At or within the irrigation device or at some point between the fluid supply or waste receptacle and the irrigation device, the separate tubing can continue through the device as three individual passageways, or can be converged into a single fluid passageway, which leads to a cooperating endoscope.

The fluid passageway(s) within the irrigation device lead to an exit port of the irrigation device, which is connected directly or indirectly to a conventional endoscope. Regardless of the number of passageways leading from the irrigation device to the endoscope, the endoscope may have one or more channels for transporting fluids. For example, if three channels exit the irrigation device, the channels can converge into a single channel of the endoscope or can continue as three or fewer separate channels of the endoscope. The irrigation device can be connected to the endoscope by an adapter. Typically, such an adapter can comprise tubing and can for example be Y-shaped to allow for the introduction of auxiliary instruments during surgery through a second channel.

The irrigation devices according to the invention can be configured to be compatible with any conventional endoscope, including any gastroscope, colonoscope, sigmoidoscope, or enteroscope, to name a few. For example, the irrigation devices can be used with gastroscopes manufactured by Olympus under the GIF-series, including the Olympus GIF-100, GIF-1T100, GIF-1T140, GIF-2T100, and GIF-XTQ160; and gastroscopes manufactured by Pentax under the EG- or FG-series, including Pentax EG-1540, EG-3800T, EG-2930K, or FG-24X to name a few. The irrigation devices can be used with colonoscopes, including Olympus CF- or PCF-series colonoscopes, for example, Olympus CF-100L, 100-TL, 140L, 1T100L, or 20L, and including Pentax EC-series colonoscopes, for example, Pentax EC-3400F, 3400L, 3430L, 3801L, and 3872LK to name a few. Further, the irrigation devices can be compatible with sigmoidoscopes, including any of the Olympus CF-, P-, OSF-, or FS-series, including for example Olympus CF-100S, CF-140S, P10S, OSF, OSF-2, OSF-3, FS-34P, or FS-34P2. Enteroscopes that the irrigation devices can be compatible with include the Olympus SIF-100 enteroscope.

Means for connecting the exit port of the irrigation device to the endoscope can comprise any known connecting means, especially means having a quick-connect/disconnect feature. Connecting means for attaching the irrigation device to the endoscope is preferably configured so as to communicate with and establish a leak-resistant seal with the auxiliary tool port of the endoscope to allow for the delivery and aspiration of fluids such as saline, water, carbon dioxide, air, and other fluids and matter through the endoscope into and out of the patient. The connecting means need not require a direct connection between the irrigation device and the endoscope. For example, the components of the system may be connected by an intermediate structure (adapter), which comprises any combination of tubing and/or connectors. The adapter can comprise quick connect/disconnect features at the connecting ends so as to facilitate connection with the irrigation device at one end and the endoscope at the other. Further, the adapter can comprise one or more additional ports or channels for therapeutic instruments to be used in conjunction with the system, such as a Y-shaped adapter, including asymmetric Y-shaped adapters.

In embodiments, the fluids are supplied to the irrigation device under pressure. The irrigation fluid source can be a saline drip in which gravity is used to deliver the saline into the patient. If more fluid pressure is desired, however, a pump can be included with the system to deliver the saline from the saline source. A pump can be used alone or in combination with a saline drip or the two can be used concurrently or alternatively. The saline is typically delivered in relatively large volumes at a low velocity but can also be delivered in low volumes at a high velocity, depending on the pressure supply. Different surgical circumstances may call for different fluid functions, such as using a high volume of saline to wash out matter from a patient or dilute matter within the patient, whereas low volumes in relatively high velocity streams can be used to break up larger material and debris or focus the saline stream in a certain place.

In embodiments, saline or other washing fluid such as water can be supplied under pump pressure. Preferably, an electric-driven pump with either portable or fixed electricity is used to deliver saline through the irrigation device and system. Any conventional pump capable of providing appropriate fluid pressure can be used. The pump can additionally comprise a regulator for controlling the maximum fluid pressure for the system.

The irrigation devices and systems according to the invention provide for the delivery of pressurized gas, for example, carbon dioxide or air, in cases where it is desired to insufflate a cavity within the patient. Such insufflation functionality can be beneficial in situations where the surgeon desires to move folded tissue out of the way, which may be obstructing the surgical site or which may itself constitute the bleeding or surgical site. Pressurized gas such as air or carbon dioxide, can be delivered to the patient to temporarily insufflate the cavity. As with the delivery of wash fluid into the patient, the source of gas is also provided under pressure. A pump, however, is typically not required to deliver the gas source, as the gas is typically stored and contained within a pressurized housing. Thus, the gas is automatically delivered under pressure when released from the container. To be sure that an appropriate amount of pressure and/or gas is delivered, a regulator may be used in conjunction with delivering the pressurized gas source through the irrigation device and system. Although air can be used to insufflate a cavity within the patient, carbon dioxide may be preferred in some circumstances because it is more readily absorbed by the body through the gastrointestinal tract.

If a pump is used for aspirating or suctioning fluid and debris from a patient, any conventional pump known in the art can be used. For example, the pump can be provided as an in-wall vacuum system as typically supplied by a hospital or an electric-driven pump having a source of power such as a battery and/or means for connecting the pump to an electrical supply. In embodiments, the same pump used for introducing fluids into the patient can be used for removing fluids and debris from the patient.

The irrigation device is controllable by, preferably, a hand-held device with controls for directing the flow and/or aspiration of fluids. For example, the hand-held portion can be ergonomically shaped. It can be comprised of plastic or metal. The hand-held device comprises keys (e.g., push buttons), mechanical means for controlling fluid flow through the irrigation system, e.g., valves for stopping fluid flow by obstructing the tubing channels with some structure and for starting fluid flow by moving the obstruction out of the fluid path. Fluid is caused to move through the channels by way of, for example, pumps. In one embodiment, a pump can be used to cause the saline from the saline source to be imported into and through the irrigation device. Alternatively, or concurrently, the saline can be moved through the system by using a bag or other container to hold the saline at a height (for example, above the patient) that would allow for gravity to impose a pressure on the fluid, thus, moving it through the tubing.

The mechanical buttons for controlling fluid flow preferably operate independently of one another and can be operated simultaneously or individually. For example, carbon dioxide and saline can be simultaneously or consecutively introduced to the patient through this device. Likewise, matter (carbon dioxide, air, saline, blood, debris, etc.) can be removed from the patient by aspiration before, after, or even during introduction of additional fluids into the patient.

In embodiments, one or more pressure sensors can be incorporated into the device or system to monitor and/or control pressure within the patient's body. Too much fluid introduced into the patient can potentially cause injury/damage to the patient. As an example, if a surgeon were operating the device to clear a patient's stomach, there may be a certain pressure the patient's stomach would tolerate from pressure caused by the introduction of fluid into the patient's body. Pressure monitors could be used to deliver a warning to the surgeon that internal pressure of the patient is at a dangerous level and/or be used to automatically shut down the system when pressure is at a critical level.

### BRIEF DESCRIPTION OF THE DRAWINGS

The accompanying drawings, which are incorporated in and constitute a part of this specification, illustrate several embodiments of the invention. Together with the written description, these representative embodiments serve to explain certain principles or details of various aspects of the present invention.

FIG. 1 shows a representative irrigation device.

FIGS. 2A, 2B, 2C, and 2D show various views of a representative irrigation device.

FIG. 3 shows a base supply unit for a representative irrigation system.

FIG. 4 shows a representative conventional endoscope.

FIG. 5 shows a detailed view of a port for a conventional endoscope.

FIG. 6 shows a detailed view of the insertion tube of a conventional endoscope.

FIGS. 7A, 7B, 7C, and 7D show representative structure for means for connecting the irrigation device to the base supply unit and/or the endoscope by way of a quick-connect/disconnect feature.

FIG. 8 shows representative intermediate structure for connecting the irrigation device to the endoscope, e.g., a Y-shaped adapter.

### DETAILED DESCRIPTION OF VARIOUS EMBODIMENTS OF THE INVENTION

Reference will now be made in detail to various exemplary embodiments of the invention, examples of which may also be illustrated in the accompanying drawings. The following detailed description is provided to give the reader a better understanding of certain features and details of embodiments of the invention, and is not to be understood as a limitation on any aspect or feature of the invention as broadly disclosed herein, depicted in the figures, or claimed. It will be readily apparent to those of skill in the art that various other modifications to the present invention may be made without departing from the scope and spirit of the invention.

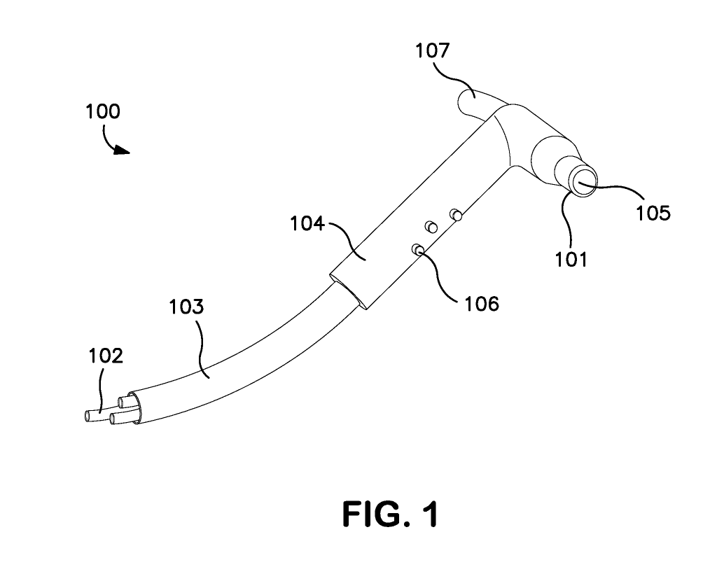

FIG. 1 shows a representative irrigation device (100) according to the invention. As used in the context of this application the term “irrigation device” is used to refer to any auxiliary surgical instrument with multi-function capabilities, including suction, irrigation, and insufflation functions, for use concurrently with an endoscope. For convenience, such auxiliary devices according to the invention may be referred to in this application simply by any one or more of the multi-function capabilities. For example, the auxiliary devices may be referred to simply as irrigation devices, even though the irrigation devices have other capabilities in addition to the irrigation functionality.

As shown, irrigation device (100) is a hand-held device for use concurrently with an endoscope. Conventional endoscopes typically comprise a port for the connection and interaction with other component instruments. The irrigation device is configured to connect indirectly or directly, conveniently, and quickly to the component instrument port of a corresponding endoscope by way of a quick-connect mechanism incorporated into exit port (101) of the irrigation device. Any standard connecting mechanism will suffice. Connections that provide for ease of use, a leak-resistant, preferably leak-proof, seal between the endoscope and the irrigation device, and which have rapid connect/disconnect capabilities are preferred. Any male/female fitting, e.g., such as a screw and counter-screw combination, barbed, flare, compression, push-to-connect, and cam fittings to name a few, will suffice. The irrigation device may be connected indirectly to the endoscope, for example, by way of intermediate structure. In embodiments, the means for connecting the intermediate structure to the endoscope and irrigation device can comprise tubing having connectors at either end to connect to the device and endoscope, or the means for connecting can be incorporated into the endoscope and/or the irrigations device, or can be stand alone.

The irrigation device is controlled by a surgeon in one hand, while the connected endoscope is operated and controlled by the surgeon in the other hand. An advantage to connecting the irrigation device directly to the endoscope is that all functions of the surgery can be performed through one tube that is inserted into the patient, namely the insertion tube of the endoscope. Thus, there is no need for multiple tubes to be inserted into the patient and the surgeon is not required to insert, remove, and reinsert the instruments used during surgery because all instrument functions are operated by the surgeon through the insertion tube of the endoscope. The single tube extending from the endoscope preferably comprises one or more channels for delivering and aspirating fluid and for supplying a camera for viewing.

The hand-held irrigation device can be configured to have one or more channels (102) for transporting fluids into or out of the patient. Fluid channels (102) can be further comprised in a sheath (103), i.e., housing for containing and controlling the multiple channels. Sheath (103) is then connected or contained within housing (104) of the irrigation device. One of channels (102) can be used for delivering saline into a gastrointestinal cavity of a patient, such as the stomach. A second channel (102) may be used for aspirating or evacuating fluids and other matter from the patient, including blood and clots, or saline and gas introduced into the patient. A third fluid channel (102) may be used to deliver pressurized gas, such as air or carbon dioxide, into the patient to insufflate the patient's internal cavity for improved viewing at the surgical site. Additionally, a single channel may be dedicated to a single fluid or may be used to transport more than one fluid or perform more than one fluid function, such as transporting saline and aspirating fluids.

Fluid flow within channels (102) is controlled by the surgeon preferably by way of finger-tip controls. In this embodiment, three push-button controls (106) provide means for controlling fluid through fluid channels (102). Controls (106) can comprise any type of valve and valve control system for allowing fluids to flow in or out of the hand-held device.

Depending on the number of channels in the cooperating endoscope, fluid channels (102) of the hand-held device can converge at controls (106) into one or more corresponding transition channels (105). Transition channels (105) lead from the irrigation device to the cooperating endoscope. Although, e.g., three channels (102) may be used to deliver/remove fluids into/out of the irrigation device from/to the fluid supply/waste storage, one transition channel (105) could be used to transport any of the multiple fluids to and from the patient through a single corresponding channel of the endoscope. Any conventional adapter or intermediate structure may be used to converge multiple channels (102) into transition channels (105) either within housing (104) or prior to the tubing entering housing (104). Further, channels (102) at the point of the valve system within housing (104) may empty into a single chamber, which then leads to one or more transition channels (105) out of irrigation device (100) and into a cooperating endoscope.

Advantages, however, may be achieved by having dedicated channels in both the device and the endoscope for particular fluids and/or functions, for example to be able to aspirate fluid from the patient while concurrently introducing saline into the patient for continuous cleaning of the surgical site. A single transition channel (105), however, also has advantages in that the corresponding endoscope can be less complex by having only one channel for fluid functions. Regardless of the number of channels entering and exiting the irrigation device, the irrigation device can easily be configured by one of ordinary skill in the art to achieve the appropriate goal by configuring appropriate connectors and/or adapters both within the irrigation device and with respect to the quick connect function of exit port (101).

The hand-held irrigation device can be configured so as to be comfortable and convenient for the surgeon to hold. For example, the device can comprise a pistol-type grip with structure for providing additional security for handling the device during surgery, for example by providing rubber or other grip-enhancing material to contact the surgeon's palm or fingers on the grip. The hand-held device is also preferably weight balanced so as to minimize the effort required by the surgeon's grip in holding and operating the device. The device can also comprise structure for resting the device in the surgeon's hand, such as thumb rest (107), which can be used to support the instrument during use of the irrigation device between the surgeon's thumb and forefinger.

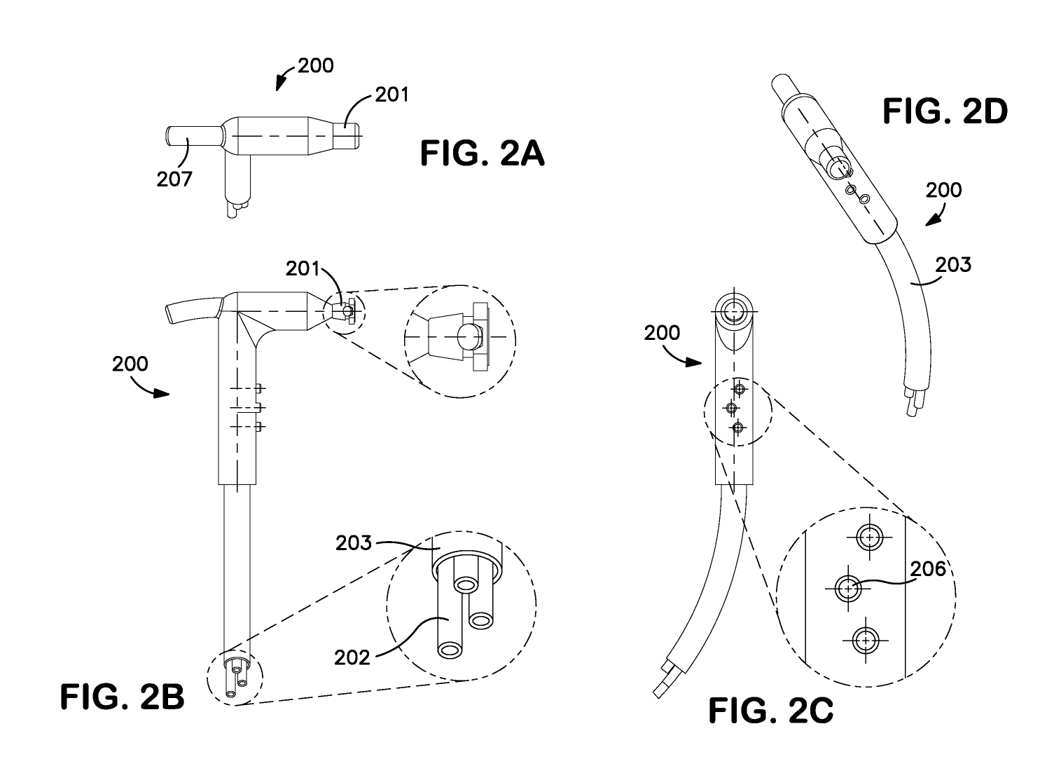

FIGS. 2A, 2B, 2C, and 2D show various views of irrigation device (200). In particular, FIG. 2A shows a top view of irrigation device (200) comprising optional support structure (207), which provides means for controlling positioning and operation of the device during surgery. Support structure (207) may also be referred to as a thumb rest, which can be cradled by the surgeon's hand between the thumb and forefinger. Thumb rest (207) provides stability and enhances control of the device during use. Also shown is exit port (201), which provides means for connecting the irrigation device to an endoscope. Exit port (201) can be connected directly to an endoscope by comprising structure for such direct connection or can be connected to the endoscope indirectly by cooperating with intermediate structure, such as adapters (which can comprise any combination of tubing, fittings, or connectors).

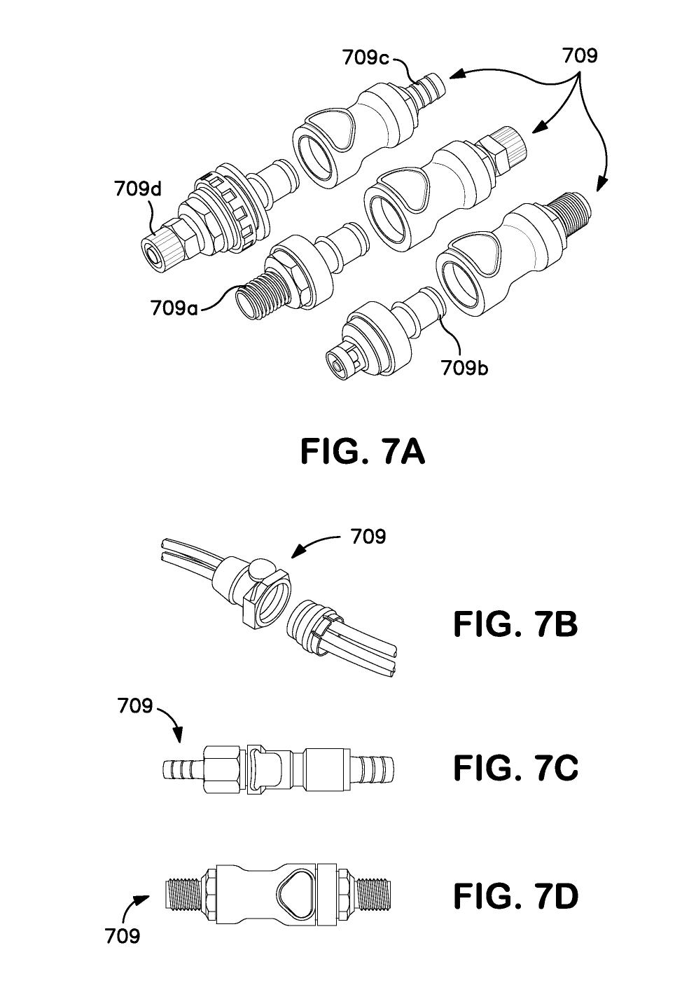

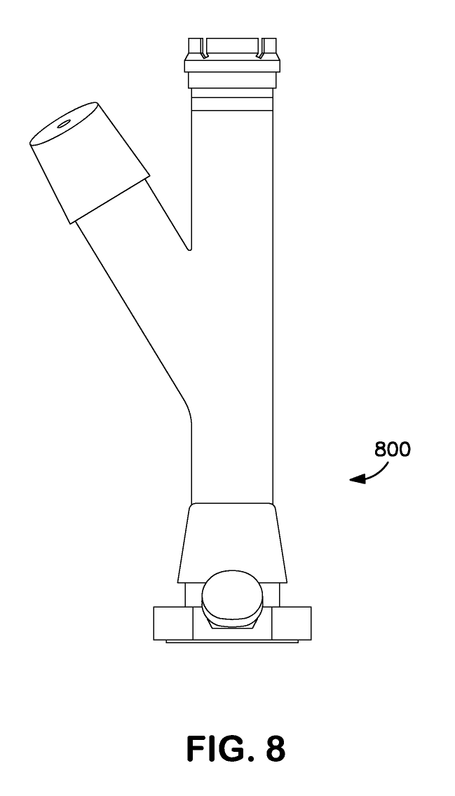

FIG. 2B shows a profile view of irrigation device (200). Irrigation device (200), as shown, can conform to a generally pistol-type grip but can also conform to any shape convenient and comfortable for a surgeon to use. The instrument will preferably comprise an ergonomically-shaped device that is substantially rigid, for example comprised of hard plastic or metal. As shown, the overall shape of the device housing can comprise approximately a right angle. The housing may also be configured to comprise an overall shape of greater than or less than a right angle and additional advantages may be achieved under certain circumstances with such configurations. For example, a housing comprising greater than a right angle may contribute to unimpeded fluid flow through the tubing contained within the housing due to having less of a bend in the tubing within the housing. A close-up view of exit port (201) is provided to show that exit port (201), which connects indirectly or directly with an endoscope, can form part of the housing of the device and can, for example, connect to the endoscope by being inserted into the auxiliary instrument port of the endoscope or the auxiliary instrument port can be inserted into port (201) of the irrigation device. Port (201) may also connect to the endoscope by way of intermediate structure, for example, structure substantially comprising tubing having means for communicating with exit port (201) and a cooperating endoscope. In this embodiment, exit port (201) of the irrigation device is configured with a quick-connect/disconnect fitting to provide for connection with the endoscope or intermediate structure. The connection feature incorporated into the irrigation device can be of any configuration (e.g., male or female) so long as the endoscope and/or intermediate structure comprise structure for cooperating with the connecting feature of the irrigation device. Examples of such connecting means can be found in FIGS. 7 and 8. Means for connecting the irrigation device and/or the intermediate structure to the endoscope can be incorporated into any of the components of the system or can be stand alone.

A close-up view of sheath (203) comprising tubing channels (202) is further provided by FIG. 2B. Sheath (203) is optional for containing and controlling tubing channels (202), which lead from the fluid and suction supplies to the hand-held irrigation device. Sheath (203) can comprise any flexible material, such as fabric, rubber, plastic, or metal mesh to name a few. Tubing channels (202) lead individually from the fluid and suction supplies to the irrigation device (200), however, the number of tubing channels (202) can be reduced from three to one, if desired, at any point in the system, including at the fluid or suction supply, at the irrigation device, and/or at the endoscope. For example, within the irrigation device at the valves, tubing channels (202) can converge into one chamber/channel. A single channel passing through irrigation device (200) may be advantageous in reducing the complexity of irrigation device (200), which can cooperate with similarly less complex endoscopes, e.g., endoscopes with only one fluid channel.

FIG. 2C shows representative means for controlling fluid flow through irrigation device (200). As shown, such means can be provided by push buttons (206), which when depressed will permit flow of one or more of the suction, carbon dioxide, and/or saline functions. Likewise, when released, push buttons (206) will suspend fluid flow. Buttons or keys (206) are preferably mechanical in nature and can comprise a valve system for controlling fluid flow through the device. Additionally, buttons (206) preferably operate individually and/or cooperatively to provide one or more functions concurrently.

FIG. 2D shows a representative irrigation device (200) having flexibility in sheath (203), which contains the tubing for transportation of fluids from the fluid supply to the endoscope and from the endoscope to waste.

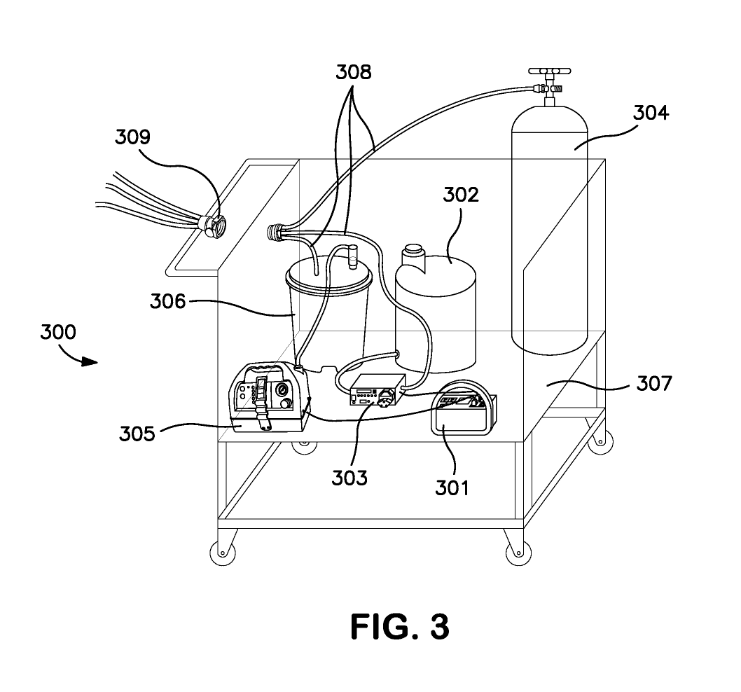

FIG. 3 shows a representative base unit (300), which can be combined with the irrigation device and an endoscope to comprise an endoscopic irrigation system according to the present invention. Base unit (300), as shown, is preferably portable. Base unit (300) can comprise power source(s) (301), such as a battery, or can be configured to plug into a separate electrical source, such as a wall outlet. Additionally, any component of base unit (300), which requires electricity, can comprise its own source of electricity. For example, the pump(s) for providing irrigation fluid and/or the suction source may comprise their own source of power, such as by incorporating a portable suction unit into base unit (300). Portability of base unit (300) can be further enhanced by having base unit (300) comprise its own source(s) of power.

Base unit (300) further comprises an irrigation fluid supply (302). Fluid supply (302) can comprise a container or reservoir for storing liquid. The container can be rigid, for example made of hard plastic, or the container can be flexible, for example comprising a bag or other reservoir typically used for storing saline or water, such as containers for saline drip. Exporting the irrigation fluid from fluid supply (302) to the irrigation device can be achieved by having tubing reach the bottom of the container or by having an exit port at the bottom of the container, which communicates with tubing extending to the irrigation device. Fluid supply (302) is preferably incorporated into base unit (300), however, base unit (300) can also be configured to connect with an external source of irrigation fluid. Again, portability of base unit (300) may be enhanced by incorporation of a fluid source (302) into base unit (300).

A pump (303) is preferably included with base unit (300) to provide means for delivering the irrigation fluid from fluid supply (302) through the device and endoscope to the patient. Any pump for transporting fluids may be used. The pump (303) may be used alone or in combination with other means for moving fluids, such as by way of saline drip. A saline drip may provide sufficient fluid pressure under certain circumstances, e.g., if only low pressure fluid is needed. A pump (303), however, may be advantageous in certain circumstances for providing blasts of wash fluid to break up large clots or to clean the surgical site rapidly. In embodiments, pump (303) and fluid supply (302) can comprise an otherwise stand-alone pump and fluid supply that is incorporated into base unit (300) and has its own source of power.

Base unit (300) further comprises a source for pressurized gas (304). To enhance portability, the pressurized gas supply (304) can be incorporated into base unit (300). Pressurized gas supply (304) can also be supplied by an external source to which base unit (300) is connected to and cooperates with, such as a wall supply of gas typically found in hospitals. The type of gas can be any gas under pressure, such as carbon dioxide or air. Pressurized gas itself provides sufficient pressure for the surgeon without the need for a separate pump to deliver the gas. The pressure of the gas can be controlled by any type of regulator or other valve system, including or in addition to the valve system of the hand-held irrigation device. For example, to prevent gas from entering the patient at too high a pressure, one pressure regulator can be used at the gas source (304) to set a maximum pressure, while the flow control can be adjusted at the fingertip controls of the hand-held device, such as by having means for regulating fluid flow in addition to means for stopping and starting fluid flow. Having more than one regulator for controlling fluid flow of the gas, or any of the fluids transported by way of the invention, is preferred.

Base unit (300) additionally comprises a suction source (305) and waste reservoir (306). Suction source (305) can comprise any conventional pump or other vacuum means. Suction source (305) can be incorporated into base unit (300) or can be supplied from an external source, such as by way of a typical wall-embedded vacuum source found in hospital settings. Suction source (305) preferably comprises a regulator for adjusting and controlling fluid flow. Fluid flow can additionally be controlled by means for controlling fluid flow on the hand-held device. Waste reservoir (306) can comprise any container for storing waste fluid, debris, and other matter removed from the patient. More particularly, waste reservoir (306) is configured to cooperate with suction source (305) so as to receive waste aspirated through an endoscope and irrigation device by suction force supplied by suction source (305). For example, suction source (305) may interact with waste reservoir (306) by way of intermediate tubing entering at a first port at the top of the container. A separate second port at the top of the container would connect separate tubing (308) to the irrigation device. According to such a configuration, suction source (305) creates a vacuum in waste reservoir (306) through the first port, while fluid and debris are suctioned from the patient through the irrigation device and cooperating tubing into the second port of waste reservoir (306), thus, emptying the fluids and debris into the bottom of the reservoir. Fluids and debris would avoid contact with suction source (305), so long as the fluid level in the container remains below the level of the first port, the suction port. An automatic shut off feature or other warning mechanism could be incorporated into the suction system to avoid fluids reaching the first port and being suctioned into suction pump (305).

A pump can be used as the suction source and, in embodiments, the suction pump and the saline pump may comprise a single pump having multi-channel pumping capability. Suction source (305) and waste reservoir (306) are preferably incorporated into base unit (300) for increased system portability. One or more of the components of the system can be contained in, transported on, or incorporated into any structure which enhances portability of the system, such as a cart (307).

Fluids are transported to the irrigation device from fluid source (302) and gas source (304) by way of fluid channels (308). Likewise, fluids, debris, and other wastes are transported to suction canister (306) from the irrigation device by way of a third fluid channel (308). Fluid channels (308) can comprise any flexible tubing-type structure, such as plastic tubing. In this embodiment, fluid channels (308) are connected to the irrigation device by way of a multi-tube connector (309). The fluid channels of the irrigation device can also be connected directly to each fluid source and/or the vacuum. A quick-connect/disconnect fitting, such as multi-tube connector (309) has advantages in certain applications where it is desirable to be capable of assembling the system easily and quickly.

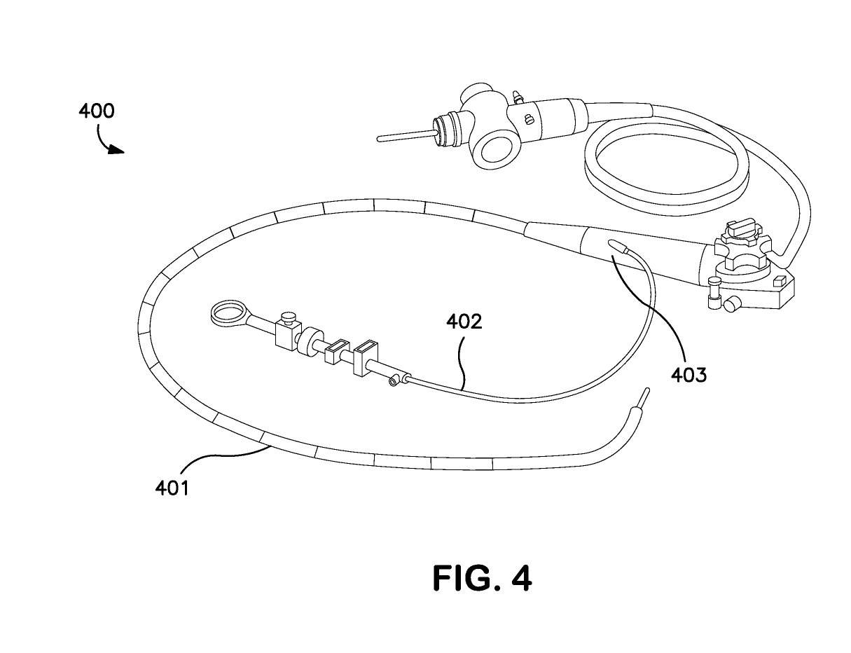

FIG. 4 shows a conventional endoscope (400), more particularly, a gastroscope. A gastroscope is used for purposes of exemplifying the invention, however, any endoscope may be used with the irrigation devices of the present invention. As shown, endoscope (400) comprises insertion tube (401), which is the tube inserted into a patient through the throat to reach part of the patient's gastrointestinal tract, such as the stomach. Insertion tube (401) comprises a camera for internal viewing of a gastrointestinal tract. Also comprised within insertion tube (401) are one or more channels for operating auxiliary tools, such as biopsy forceps (402) and/or cauterization tools. Auxiliary tools are inserted through instrument port (403). Instrument port (403) can also perform other functions, such as providing suction at the surgical site. The irrigation devices of the present invention are configured to cooperate with instrument port (403) of the endoscope. The irrigation devices of the present invention are configured to connect with instrument port (403) by any means that provides for a leak-resistant seal. Preferably, the irrigation devices are secured to the endoscopes by a quick-connect mechanism that provides for easy and rapid connection and disconnection of the irrigation device and endoscope. The quick-connect/disconnect mechanism can comprise tubing with connectors on either end for joining the irrigation device with the endoscope.

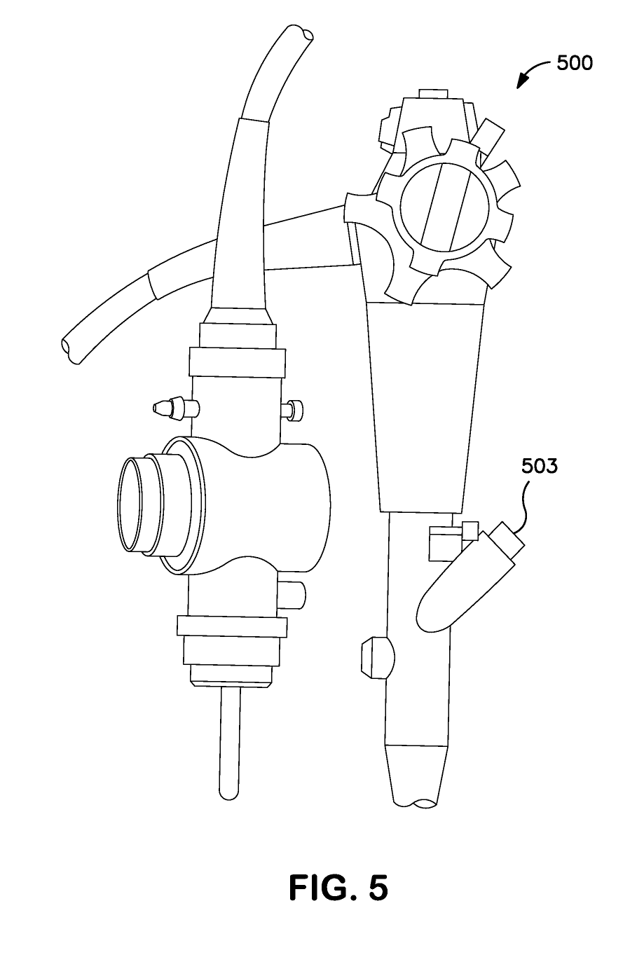

FIG. 5 shows a close-up view of various portions of a typical endoscope (500). More particularly, the irrigation devices of the present invention are configured to cooperate with instrument port (503) of the endoscopes. For example, an irrigation device could be configured with a female-type exit port that mates with a male-type instrument port (503) of the endoscope, or vice versa. Further, an adapter (such as any combination of tubing channels, fittings, and connectors) could be used to connect instrument port (503) to the irrigation device. Structure can be incorporated into the exit port of the irrigation device and/or the instrument port (503) of the endoscope for providing a leak-resistant seal, such as a rubber washer, O-ring, or other structure, and for providing simple connect/disconnect, such as a quick-release connection typically associated with connecting a hose to a water source, or connecting tubing to tubing, or connecting tubing to some other structure.

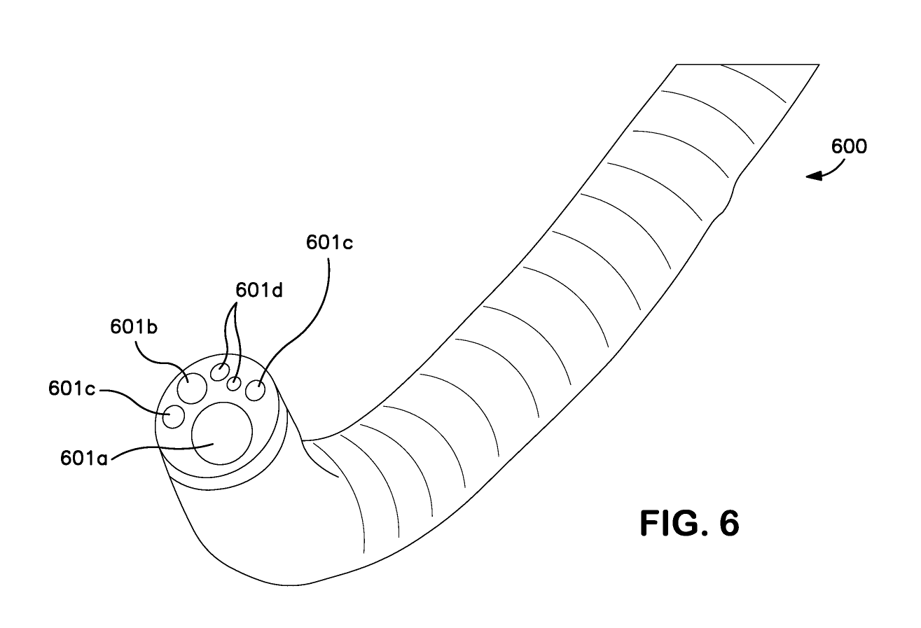

FIG. 6 shows a close-up view of an insertion tube (601) of a representative conventional endoscope (600). As shown, insertion tube (601) comprises channel (601a) typically used for introducing auxiliary instruments into a patient, such as biopsy forceps and/or cauterization tools. Some endoscopes comprise more than one such channel (601a). Endoscopes comprising one channel (601a) are typically referred to as diagnostic endoscopes, and those comprising multiple channels are typically referred to as therapeutic endoscopes. The irrigation devices according to the invention are configured so as to deliver or remove fluids from a patient through channels (601a) of an endoscope. With one channel (601a) of an endoscope, a cooperating irrigation device can transport one or multiple fluid(s) in or out of a patient through channel (601a). Advantages are achieved, however, with endoscopes comprising more than one channel (601a) so that the multiple functionalities of the irrigation device can be performed concurrently. For example, with at least two channels (601a) in an endoscope, a corresponding irrigation device could concurrently deliver saline to the surgical site, while suctioning the site, so that continuous cleaning of the surgical site can be performed. Likewise, if continuous insufflation is needed while cleaning, one channel (601a) could be used to deliver pressurized gas, while another channel (601a) concurrently is used to deliver saline and/or suction the saline from the site. The number of channels (601a) of the endoscope is not critical and the corresponding irrigation device can easily be adapted for use with multiple types of endoscopes. Also shown in FIG. 6 is scope (601b), a camera for viewing the surgical site within the gastrointestinal tract; light sources (601c) to assist with viewing through camera (601b); and channels (601d) for providing small amounts of water and suction for cleaning camera (601b) in situations where the view through the lens is obstructed. The water and suction sources provided by existing endoscopes are not capable of the rapid irrigation functions provided by the present invention. For example, the weak suction source in combination with the small diameter of channel (601d) would be incapable of evacuating large clots from a patient as well as incapable of evacuating a gastrointestinal cavity sufficiently and quickly.

FIGS. 7A, 7B, 7C, and 7D show representative fittings for connecting the irrigation device to the endoscope and for connecting the irrigation device to the fluid sources and/or source of suction/waste. Provided in FIG. 7A, for example are multiple types of connectors, each having a quick-connect/disconnect feature. Many variations exist for connecting the connectors with tubing, the irrigation device, and the endoscope, including as exemplified the screw-type (709a), the male/female with O-ring type (709b), the male/female barbed type (709c), and the compression fitting (709d). Connectors (709) can be incorporated into any piece of the system or can be stand alone. For example, the exit port of the irrigation device itself can be configured as half of a quick-connect/disconnect structure. Likewise, connector (709) can be used in conjunction with or incorporated into intermediate structure (adapter) for connecting the irrigation device to the endoscope, or can be incorporated into the irrigation device and/or endoscope. FIG. 7B shows a representative multi-tube connector, which can be used for connecting multiple fluid channels within the system. FIG. 7C provides a barbed-type connector, which also acts as a reducer to transition from one size fluid channel to a smaller or larger size fluid channel. FIG. 7D provides another example of a quick-connect/disconnect feature, which comprises screw-type fittings.

FIG. 8 provides a representative Y-shaped adapter (800) for connecting the irrigation device to the endoscope. The adapters can be of any substantially cylindrical shape, including a single-channel cylinder. Further, for example, as shown in FIG. 8, an asymmetric Y-shaped cylinder can also be used. An asymmetric Y-shaped adapter would comprise a first channel that is substantially linear and a second channel diverging from the first channel. Y-shaped adapters are desirable for applications where auxiliary instruments are to be used through the endoscope. For example, irrigation, suction, and insufflation can be provided through one channel of the Y-shaped adapter, such as the main substantially linear channel, while auxiliary instruments, such as biopsy forceps and/or cauterization instruments can be used through the other channel. Intermediate structure, adapters (800), can be configured to have any means for connecting the adapter to the irrigation device and the endoscope. In embodiments, kits are provided comprising multiple adapters and/or multiple fittings. As shown in this embodiment, one end of the main channel of the adapter (800) can comprise a quick-connect/disconnect feature for connecting with a cooperating feature of the irrigation device and the other end of the main channel can comprise similar structure for cooperation with an endoscope. Other intermediate structure can be used between the endoscope and the Y-shaped adapter, for example, to increase the distance between the endoscope and the auxiliary tools so that operation of the auxiliary tools is not impeded by the endoscope because of limited working space. Also, as shown, the auxiliary channel can comprise a port for accommodating auxiliary instruments, such as biopsy forceps, which is further equipped with means for preventing fluids from exiting that port, such as a rubber port that cooperates snugly with the auxiliary instruments to allow the instruments to be introduced to the channel, while preventing fluids from exiting that channel. A cap or other means for preventing fluid from exiting the auxiliary port when not in use can also be incorporated.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. An irrigation device for endoscopic surgery comprising:
a handle comprising valves for controlling fluid flow;
at least one channel for transporting fluid; and
a connection port adapted to communicate with an endoscope for delivering fluid to and receiving fluid from said endoscope through said at least one channel.

2. The irrigation device according to claim 1, further comprising intermediate structure for communicating said irrigation device connection port to said endoscope.

3. The irrigation device according to claim 2, wherein said intermediate structure comprises a Y-shaped adapter for providing instrument access through said endoscope.

4. The irrigation device according to claim 1 comprising at least three channels for transporting fluid from a fluid source or transporting fluid to a waste container.

5. The irrigation device according to claim 4, wherein a first channel is capable of transporting irrigation fluid, a second channel is capable of transporting pressurized gas, and a third channel is capable of transporting aspirated fluid.

6. The irrigation device according to claim 5, wherein said irrigation fluid is saline and said pressurized gas is carbon dioxide.

7. The irrigation device according to claim 1 further comprising at least one pressure sensor for monitoring fluid pressure.

8. A system for endoscopic surgery or irrigation comprising:
an irrigation device comprising:
a handle comprising valves for controlling fluid flow;
at least one channel for transporting fluid; and
a connection port adapted to communicate with an endoscope for delivering fluid to and receiving fluid from said endoscope through said channels; and
a supply of fluid and suction.

9. The system according to claim 8 further comprising an endoscope.

10. The system according to claim 9 further comprising an intermediate structure for communicating said irrigation device connection port with said endoscope.

11. The system according to claim 10, wherein said intermediate structure comprises a Y-shaped adapter for providing instrument access through said endoscope.

12. The system according to claim 8, wherein said irrigation device is capable of transporting irrigation fluid, pressurized gas, and suction through said at least one channel for transporting fluid.

13. The system according to claim 12, wherein said irrigation fluid is saline, said pressurized gas is carbon dioxide, and said suction is a vacuum.

14. The system according to claim 8 further comprising a pressure sensor for monitoring fluid pressure.

15. The system according to claim 8, wherein said supply of fluid and suction is portable.

16. A method of providing instruments for endoscopic irrigation or surgery comprising:
providing an irrigation device comprising:
a handle comprising valves for controlling fluid flow;
at least one channel for transporting fluid; and
a connection port adapted to communicate with an endoscope for delivering fluid to and receiving fluid from said endoscope through said channels.

17. The method according to claim 16 further comprising an intermediate structure for communicating said irrigation device connection port to said endoscope.

18. The method according to claim 17, wherein said intermediate structure comprises a Y-shaped adapter for providing instrument access through said endoscope.

19. A method of performing endoscopic surgery or irrigation comprising:
providing an irrigation device comprising:
a handle comprising valves for controlling fluid flow;
at least one channel for transporting fluid; and
a connection port adapted to communicate with an endoscope for delivering fluid to and receiving fluid from said endoscope through said channels;
providing an endoscope; and
performing an endoscopic procedure.

20. The method according to claim 19, wherein the irrigation device further comprises an intermediate structure for communicating said irrigation device connection port to said endoscope.

21. The method according to claim 20, wherein said intermediate structure comprises a Y-shaped adapter for providing instrument access through said endoscope.

22. A kit for connecting an irrigation device according to claim 1 to an endoscope comprising multiple intermediate structures for communicating said irrigation device connection port to said endoscope.

23. The kit according to claim 22, wherein at least one of said multiple structures comprises a Y-shaped adapter for providing instrument access through said endoscope.
